(19) United States
(12) Patent Application Publication
(10) Pub. No.: US 2020/0273556 A1
(43) Pub. Date: Aug. 27, 2020

(54) METHOD OF PROVIDING HEALTH MANAGING SERVICE LINKED TO EXERCISE EXAMINATIONS, AND SERVER DEVICE FOR SAME

(71) Applicant: FITT Corp., Seongnam-si, Gyeonggi-do (KR)

(72) Inventor: Suk Jea HONG, Seongnam-si, Gyeonggi-do (KR)

(21) Appl. No.: 15/781,023

(22) PCT Filed: Nov. 28, 2016
(86) PCT No.: PCT/KR2016/013750

§ 371 Date: Jun. 1, 2018

(30) Foreign Application Priority Data
Dec. 4, 2015 (KR) 10-2015-0172210
Dec. 4, 2015 (KR) 10-2015-0172217

(51) Int. Cl. G16H 20/30; G16H 20/60; G16H 10/60; G16H 40/67; G16H 40/63; G16H 50/30; G16H 50/20; G16H 80/00; G16H 15/00

(52) U.S. Cl. CPC G16H 20/30; G16H 20/60; G16H 10/60; G16H 40/67; G16H 15/00; G16H 50/30; G16H 50/20; G16H 80/00; G16H 40/63

(57) ABSTRACT

Provided are a method of providing a health management service while operating in conjunction with an exercise test and a server apparatus therefor. The method includes providing information about an exercise program suitable for an examinee based on a cardiopulmonary capacity value of the examinee obtained by performing an exercise test, calculating weekly calorie consumption of the examinee based on calorie consumption and the number of exercises per week corresponding to the exercise program, determining a change in the weekly calorie consumption of the examinee based on a cardiopulmonary capacity value of the examinee obtained by performing the exercise test again after the exercise program for a specific period, and determining whether at least one of a plurality of guidelines related to the weekly calorie consumption is satisfied or an arrival ratio based on the determined change in the weekly calorie consumption of the examinee.

### 1. TECHNICAL FIELD

The present invention relates to a method of providing a service capable of managing a user's health by analyzing health records or measuring the user's physical level while operating in conjunction with an exercise test.

The present application claims the benefit of Korean Patent Application Nos. 10-2015-0172210 and 10-2015-0172217 filed in the Korean Intellectual Property Office on Dec. 4, 2015, the entire contents of which are incorporated herein by reference.

### Background of the Invention

### 2. DESCRIPTION OF THE RELATED ART

With the development of the electronic technology, various types of electronic devices are developed and supplied. Specifically, various types of display devices, such as TV, a mobile phone, a tablet PC, a laptop PC, and a PC, are used a lot. Accordingly, a user's needs to receive various services using the display device have increased.

Specifically, regarding various exercises performed in the fitness center, recently, a user can be provided with various services including an exercise map through a web or mobile.

For example, Korean Patent Application Publication No. 2013-89765 discloses “Personal exercise management system for using affiliated fitness center and method thereof.” The disclosed exercise management system includes an exercise management server, including an automatic member subscription unit for automatically registering a user who becomes a member of an affiliated fitness center with a website, an entrance/exit management unit for receiving entrance/exit information of a member for an affiliated fitness center, an instructor terminal notification unit for notifying an instructor mobile terminal of information about an entered member, an instructor exercise map result registration unit for registering exercise map results and physical change results from the instructor mobile terminal; and a member exercise result registration unit for registering the exercise results of a member from a user terminal. Accordingly, although a member visits a specific affiliated fitness center, the member can be provided with an instructor's exercise map service suitable for his or her exercise program based on exercise information of the member transmitted to the instructor in real time.

Furthermore, attempts to provide exercise-related services to a user through a mobile app or wearable device instead of a trainer continue to be made, but the exercise-related services have a problem in that service utilization is low because they detect a user's motion and provide only fragmentary exercise effects, such as exercise results and corresponding calorie consumption.

### SUMMARY OF THE INVENTION

Accordingly, an embodiment of the present invention provides a method of providing a health management service while operating in conjunction with an exercise test, which can prevent diseases effectively by measuring a user's physical level, and a server apparatus therefor.

Furthermore, an embodiment of the present invention provides a method of providing a health record analysis service while operating in conjunction with an exercise test, which can induce a user's exercise effectively by analyzing health records, and a server apparatus therefor.

A method of providing a health management service while operating in conjunction with an exercise test according to an embodiment of the present invention includes providing information about an exercise program suitable for an examinee based on a cardiopulmonary capacity value of the examinee obtained by performing an exercise test, calculating weekly calorie consumption of the examinee based on calorie consumption and the number of exercises per week corresponding to the exercise program, determining a change in the weekly calorie consumption of the examinee based on a cardiopulmonary capacity value of the examinee obtained by performing the exercise test again after the exercise program for a specific period, and determining whether at least one of a plurality of guidelines related to the weekly calorie consumption is satisfied or an arrival ratio based on the determined change in the weekly calorie consumption of the examinee.

Furthermore, a server apparatus according to an embodiment of the present invention provides a health management service while operating in conjunction with an exercise test, and includes a cardiopulmonary capacity analysis unit configured to provide information about an exercise program suitable for an examinee based on a cardiopulmonary capacity value of the examinee obtained by performing an exercise test, a calorie calculation unit configured to calculate weekly calorie consumption of the examinee based on calorie consumption and the number of exercises per week corresponding to the exercise program, a change determination unit configured to determine a change in the weekly calorie consumption of the examinee based on a cardiopulmonary capacity value of the examinee obtained by performing the exercise test again after the exercise program for a specific period, and an analysis unit configured to determine whether at least one of a plurality of guidelines related to the weekly calorie consumption is satisfied or an arrival ratio based on the determined change in the weekly calorie consumption of the examinee.

The method of providing a health management service while operating in conjunction with an exercise test may be implemented as a recording medium on which a program for executing the method in a computer has been recorded.

Furthermore, a method of providing a health record analysis service while operating in conjunction with an exercise test according to an embodiment of the present invention includes providing information about an exercise program suitable for an examinee based on a cardiopulmonary capacity value of the examinee obtained by performing an exercise test, obtaining health records of the examinee, determining a change in a cardiopulmonary capacity value by comparing the cardiopulmonary capacity value of the examinee with a cardiopulmonary capacity value of the examinee obtained by performing the exercise test again after the exercise program for a specific period, determining a change in the results of medical check-up by comparing the health records of the examinee with health records of the examinee obtained by performing medical check-up again after a lapse of a specific period, estimating a change in the rate of one or more diseases based on the change in the results of medical check-up, and providing information about the estimated change in the rate of a disease in connection with at least one of the change in the cardiopulmonary capacity value and the change in the results of medical check-up.

A server according to an embodiment of the present invention provides a heal record analysis service while operating in conjunction with an exercise test, and includes a cardiopulmonary capacity analysis unit configured to provide information about an exercise program suitable for an examinee based on a cardiopulmonary capacity value of the examinee obtained by performing an exercise test, a change determination unit configured to determine a change in a cardiopulmonary capacity value by comparing the cardiopulmonary capacity value of the examinee with a cardiopulmonary capacity value of the examinee obtained by performing the exercise test again after the exercise program for a specific period and to determine a change in the results of medical check-up by comparing the health records of the examinee with health records of the examinee obtained by performing medical check-up again after a lapse of a specific period, memory configured to store a change in the results of medical check-up and a change in the rate of one or more diseases in a corresponding manner, and an information provision unit configured to provide information about a change in the rate of a disease corresponding to the change in the results of medical check-up of the examinee in connection with at least one of the change in the cardiopulmonary capacity value and the change in the results of medical check-up.

The method of providing a health record analysis service while operating in conjunction with an exercise test may be implemented as a computer-readable recording medium on which a program for executing the method in a computer has been recorded.

### BRIEF DESCRIPTION OF THE DRAWINGS

FIG. 1 is a flowchart illustrating a method of providing an exercise test service according to an embodiment of the present invention.

FIG. 2 is a diagram for illustrating an embodiment of a method for an examinee to receive test result values measured by performing a test of a specific condition.

FIG. 3 is a diagram for illustrating an embodiment of cardiopulmonary capacity indices of an examinee calculated using test result values.

FIG. 4 is a diagram for illustrating an embodiment of a level and exercise program information of an examinee provided based on a cardiopulmonary capacity index.

FIGS. 5 and 6 are diagrams illustrating examples of the configurations of a cardiopulmonary capacity test report provided as the results of an exercise test according to an embodiment of the present invention.

FIG. 7 is a schematic block diagram of the configuration of an exercise test service system according to an embodiment of the present invention.

FIG. 8 is a block diagram showing an embodiment of the configuration of a terminal device shown in FIG. 7.

FIG. 9 is a block diagram showing an embodiment of the configuration of a server apparatus shown in FIG. 7.

FIG. 10 is a block diagram showing another embodiment of the configuration of a server apparatus shown in FIG. 7.

FIG. 11 is a flowchart illustrating a method of providing a health management service while operating in conjunction with an exercise test according to an embodiment of the present invention.

FIGS. 12 to 15 are diagrams for illustrating embodiments of a method of providing a user with a change in the physical fitness level of an examinee through an exercise program and information about corresponding effects.

FIG. 16 is a block diagram showing another embodiment of the configuration of a server apparatus shown in FIG. 7.

FIG. 17 is a flowchart illustrating a method of providing a health record analysis service while operating in conjunction with an exercise test according to an embodiment of the present invention.

FIG. 18 is a diagram for illustrating an embodiment of a method of providing an examinee with the analysis of the results of a blood test.

FIG. 19 is a diagram for illustrating an embodiment of a method of providing an examinee with the analysis of the results of a urine test.

### DETAILED DESCRIPTION

Hereinafter, a method of providing a health management service while operating in conjunction with an exercise test and a server apparatus therefor according to embodiments of the present invention are described in detail with reference to the accompanying drawings.

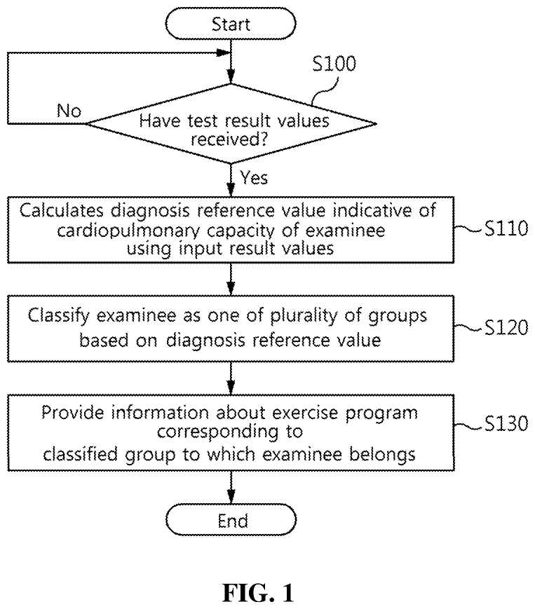

FIG. 1 is a flowchart illustrating a method of providing an exercise test service according to an embodiment of the present invention. An example in which an apparatus for providing an exercise test service performs the method of providing an exercise test service according to an embodiment of the present invention is described below, but the present invention is not limited thereto, the method of providing an exercise test service to be described below with reference to FIG. 1 may be performed by a device or two or more devices having a function other than an exercise test as a main function.

Referring to FIG. 1, the apparatus for providing an exercise test service receives test result values measured when an examinee performs a test of a specific condition (step S100).

The test may be any one of a plurality of tests previously configured to be performed under different conditions and may be for measuring the time or distance when an examinee runs a specific distance or for a specific time outdoors or an indoor exercise apparatus, such as a treadmill.

For example, a person to be tested or an examinee who performs a test may select any one of “2.4 km Run Test”, “12 min Run Test”, “M-Bruce Protocol” and “Balke Protocol” and perform a corresponding test.

“2.4 km Run Test” is an exercise test in which an examinee runs a distance of 2.4 km for the shortest time. When the run test is performed indoors, an examinee (or a person to be tested) may start at a proper speed in the treadmill, may adjust his or her pace suitable for a maximum capability value of the examinee by adjusting the speed, may run the whole course of 2.4 km, and may measure the run time.

“12 min Run Test” is an exercise test in which an examinee runs a long distance as much as possible for 12 minutes. An examinee (or a person to be tested) may start at a proper speed in the treadmill when a test is performed indoors, and may measure the running distance based on a maximum capability value of the examinee by adjusting the speed.

“M-Bruce Protocol” is to run at a slope of 11 degrees at a speed of about 5.3 km/h. An examiner may measure the time until he or she stops running while increasing the step every 1 minute as in Table 1.

TABLE 1

| Stage | 1 | 2 | 3 | 4 | 5 | 6 | 7 | 8 | 9 | 10 | 11 | 12 |
|---|---|---|---|---|---|---|---|---|---|---|---|---|
| Speed | 3.7 | 4.0 | 4.6 | 5.0 | 5.4 | 5.9 | 6.4 | 6.7 | 7.2 | 7.7 | 8.0 | 8.3 |
| (km/h) | | | | | | | | | | | | |
| Slope | 11 | 12 | 12 | 13 | 14 | 14 | 15 | 16 | 16 | 17 | 18 | 18 |

Furthermore, in “Balke Protocol”, an examinee may fix the run speed to about 5.3 km/h, and may measure the time until he or she stops running while increasing the slope from 0 degree by 1 degree per minute.

The aforementioned test method may be selected based on the exercise capability of an examinee. For example, as the exercise capability of an examinee is reduced, the order of “2.4 km Run Test”, “12 min Run Test”, “M-Bruce Protocol” and “Balke Protocol” may be applied.

The test methods are examples of a test method for providing an exercise test service according to an embodiment of the present invention, and the present invention is not limited thereto. Various exercise test methods capable of testing the cardiopulmonary capacity of an examinee may be applied to the present invention.

For example, in addition to the run test, an exercise test may be performed on an examinee through a 2000 m-rowing machine test. Specifically, after sex, an age, an experienced person, and a beginner are classified and input, an exercise test in which how fast an examinee reaches 2000 m is measured in a rowing machine, that is, an indoor adjustment exercise machine, may be performed.

Furthermore, in order to obtain a maximum capability value of an examinee, test result values may be measured through a test method suitable for the examinee in such a manner that the examinee repeatedly performs one of the test methods several times or a mixture of two or more of them.

For example, a maximum value of a test to be performed may be obtained while repeating the test several times based on a verified increment formula (e.g., increase the run speed by 0.3 km/h) using test result values measured through one test.

Accordingly, if an examinee is an experienced person, an optimal value of test result values of a test to be performed may be obtained through one or two tests. If an examinee is an inexperienced person, a more number of tests may be repeated in order to obtain test result values of the test to be performed. Furthermore, an exercise effect can be obtained by repeating the test itself.

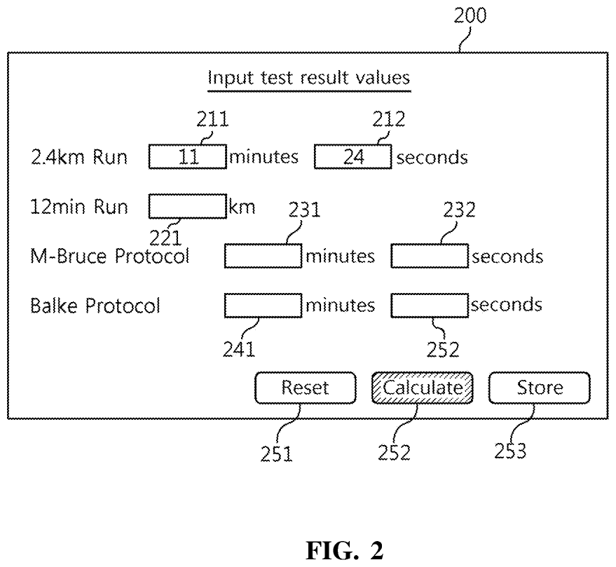

Test result values measured through the method may be displayed on a screen 200 shown in FIG. 2. Referring to FIG. 2, there are displayed test methods which may be performed by an examinee. Regions in which test result values measured by the test methods can be input may be displayed on the screen 200 of the apparatus for providing an exercise test service.

For example, if an examinee has run the whole course of 2.4 km within “11 minutes 24 seconds” as the results of the execution of “2.4 km Run Test”, the corresponding times may be displayed in “2.4 km Run Test” result value input boxes 211 and 212 on the screen 200.

Furthermore, in the screen of FIG. 2, result values displayed on the screen 200 may be reset by selecting a “Reset” button 251, the calculation of a diagnosis reference value based on input result values may be requested by selecting a “Calculation” button 252, or input result values on the screen 200 may be stored in the memory of the apparatus for providing an exercise test service or external database (DB) by selecting a “Storage” button 253.

After the test result values are received, the apparatus for providing an exercise test service calculates at least one diagnosis reference value indicative of the cardiopulmonary capacity of the examinee u sing the input result values (step S110).

For example, the apparatus for providing an exercise test service may calculate maximum oxygen consumption (VO2max) of the examinee as a diagnosis reference value using the input result values. The ratio of oxygen utilization (Mets) of the examinee may be calculated based on the calculated maximum oxygen consumption (VO2max).

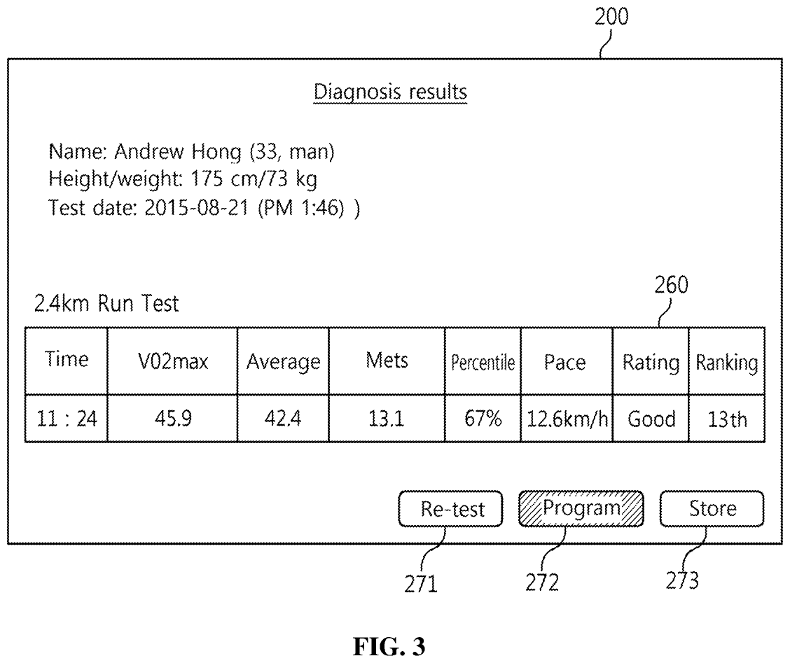

Diagnosis result values 260 calculated as described above may be displayed on the screen 200 of the apparatus for providing an exercise test service along with information of the examinee, as shown in FIG. 3, in order to indicate the cardiopulmonary capacity index of the examinee. Referring to FIG. 3, the examinee information may include a name, an age, sex, height and weight, which have been previously input before a diagnosis reference value is calculated.

Furthermore, the results of a comparison between the calculated diagnosis reference value and an age average value of the examinee may be indicated as a rank or percentage. The cardiopulmonary capacity of the examinee evaluated based on a result of the comparison may be indicated as one of “Superior”, “Excellent”, “Good”, “Fair”, “Poor” and “Very Poor.”

For example, if a percentile value calculated by comparing the maximum oxygen consumption (VO2max) of an examinee with an age average value is 95% or more, the cardiopulmonary capacity of the examinee may be indicated as “Superior.” If the percentile value is between 80% and 95%, the cardiopulmonary capacity of the examinee may be indicated as “Excellent.” If the percentile value is between 60% and 80%, the cardiopulmonary capacity of the examinee may be indicated as “Good.” If the percentile value is between 40% and 60%, the cardiopulmonary capacity of the examinee may be indicated as “Fair.” If the percentile value is between 20% and 40%, the cardiopulmonary capacity of the examinee may be indicated as “Poor.” If the percentile value is between 0% and 20%, the cardiopulmonary capacity of the examinee may be indicated as “Very Poor.”

Furthermore, the examinee may perform a test again by selecting a “re-test” button 261 on the screen of FIG. 3, and then proceed to the screen of FIG. 2 in which test result values can be entered. Alternatively, the examinee may request exercise program information optimized for the examinee based on the calculated diagnosis reference value by selecting a “Program” button 262 or may select a “Store” button 273 so that the diagnosis reference values displayed on the screen 200 are stored in the memory of the apparatus for providing an exercise test service or an external DB.

Thereafter, the apparatus for providing an exercise test service classifies the examinee as one of a plurality of groups based on the diagnosis reference value calculated at step S110 (step S120), and provides information about an exercise program corresponding to the classified group to which the examinee belongs (step S130).

That is, a plurality of groups to which the examinee may belong is present based on cardiopulmonary capacity levels. The plurality of groups has been previously set to correspond to a plurality of exercise programs having different exercise conditions.

For example, the examinee may be classified as one of “Beginner”, “Intermediate” and “Advanced” depending on that the ratio of oxygen utilization (Mets) or a maximum oxygen consumption (VO2max) value belongs to which range. Exercise programs corresponding to “Beginner”, “Intermediate” and “Advanced”, respectively, may have different exercise conditions, that is, different run speed and distances proposed to the examinee.

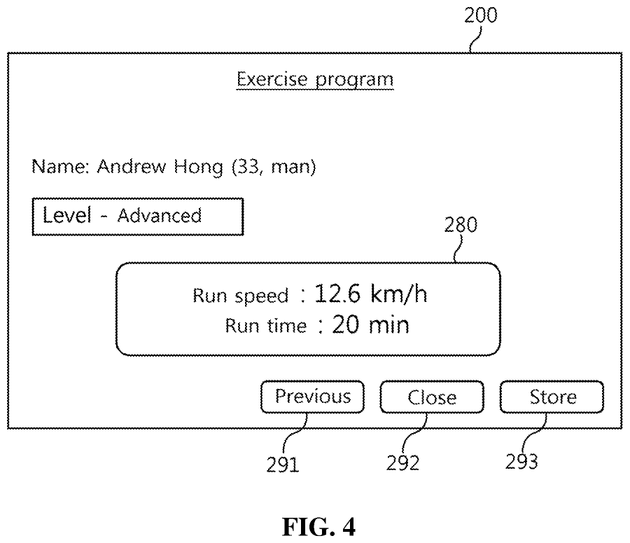

If the ratio of oxygen utilization (Mets) of an examinee calculated based on test result values is 12 or more, the cardiopulmonary capacity level of the examinee may be classified as “Advanced” and displayed on the screen 200, as shown in FIG. 4.

Furthermore, information 280 about an exercise program including exercise conditions (i.e., run speed and run time) corresponding to “Advanced” may be displayed on the screen 200 and provided to the examinee.

In accordance with an embodiment of the present invention, the run speed included in the exercise program information 280 may be determined by considering the average run speed of an examinee. The average run speed of the examinee may be a pace value calculated using test result values.

The run time included in the exercise program information 280 may be determined by considering a caloric value to be consumed through exercises.

In the screen of FIG. 4, when a “Storage” button 293 is selected, the level of the examinee displayed on the screen 200 and information about a corresponding proposed exercise program may be stored in the memory of the apparatus for providing an exercise test service or an external DB.

In accordance with another embodiment of the present invention, when test result values of an examinee have only to be input, the input result values, at least one diagnosis reference value calculated as described above and information about an exercise program corresponding to a group to which the examinee belongs may be displayed on the screen 200 or may be printed in paper and provided to the examinee (or a person to be tested).

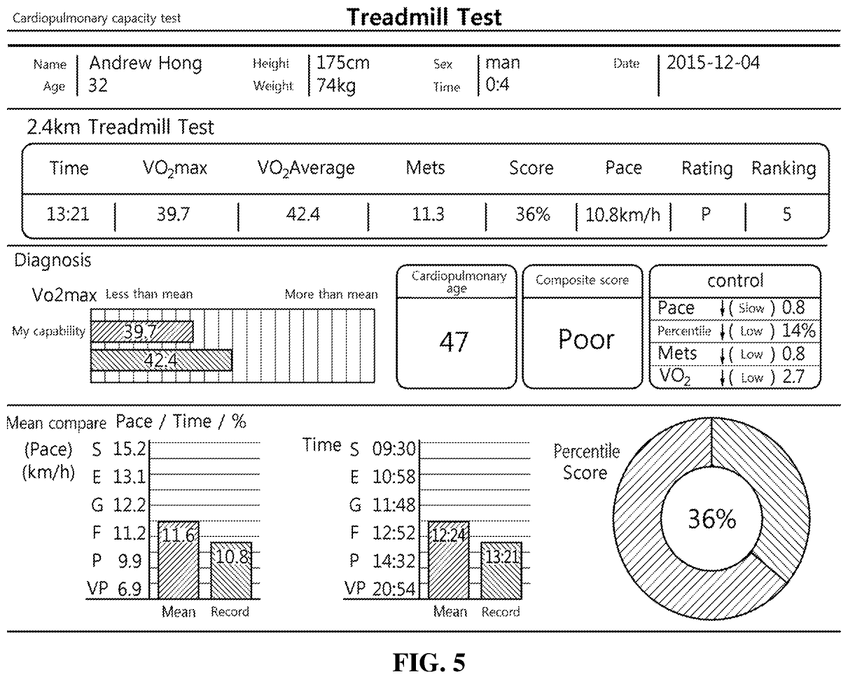

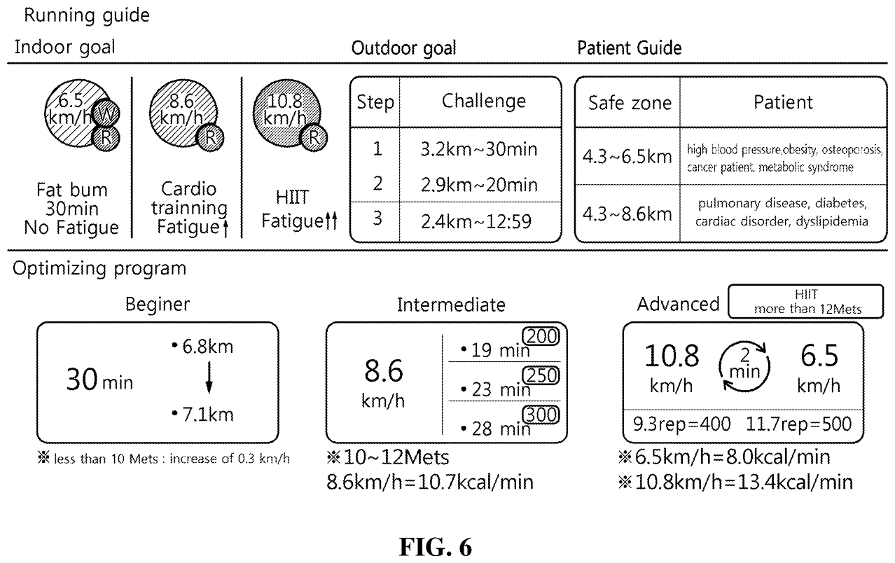

FIGS. 5 and 6 are diagrams illustrating examples of the configurations of a cardiopulmonary capacity test report provided as the results of an exercise test according to an embodiment of the present invention.

A description of configurations that belong to the configurations of the report shown in FIGS. 5 and 6 that are the same as those described with reference to FIGS. 1 to 4 are omitted hereunder. The cardiopulmonary capacity test report has been separately show in FIGS. 5 and 6, but has one sheet of report in which the contents of FIGS. 5 and 6 have been combined.

Referring to FIGS. 5 and 6, a test date and time are indicated on the upper side of the cardiopulmonary capacity test report provided to an examinee (or a person to be tested) along with the name, age, height, weight, and sex of the examinee.

The result value (Time) of a test (e.g., “2.4 km Run Test”) performed by the examinee and maximum oxygen consumption (VO2max) calculated using corresponding test result values are displayed below the examinee information along with the average value (Average) of the same sex and age as those of the examinee.

Furthermore, the ratio of oxygen utilization (Mets) calculated in proportion to maximum oxygen consumption (VO2max), percentile (Score) of maximum oxygen consumption based on the sex/age of the examinee, a pace indicative of average run speed of the examinee when a test is performed, the evaluation (Rating) of the maximum oxygen consumption (VO2max) based on the sex/age of the examinee, and a maximum oxygen consumption rank (Ranking) in the same sex and age as those of the examinee from among examinees who have perform the exercise test may be displayed as diagnosis indices indicative of the cardiopulmonary capacity of the examinee.

The cardiopulmonary capacity indices of the examinee indicated on the upper side may be visually displayed in the middle part of the cardiopulmonary capacity test report. In a “Diagnosis” item, maximum oxygen consumption (VO2max), a pace, percentile (Score), a cardiopulmonary age, and the ratio of oxygen utilization (Mets) of the examinee and the results of a comparison and difference of sex/age average values for the examinee are visually indicated.

Furthermore, in a “Mean compare” item, the results of a comparison of the pace of the examinee and time, that is, a test result value, with respect to set/age average value and percentile thereof (Score) may be visually indicated.

Information about an exercise program proposed to the examinee is indicated on the lower side of the cardiopulmonary capacity test report based on a cardiopulmonary capacity level of the examinee.

For example, in a “Run method” item, a run method suitable for the examinee is indicated based on the calculated cardiopulmonary capacity index of the examinee. The run method may include a plurality of exercise conditions based on different exercise strength or exercise steps.

More specifically, “12.6 km/h”, that is, the same run speed as a calculated pace, is indicated on the upper part as exercise (HIIT) of the highest strength. “10.1 km/h”, that is, run speed corresponding to about 80% of the pace, is indicated as exercise (cardio training) of intermediate strength. “7.2 km/h”, that is, run speed corresponding to about 60% of the pace, is indicated as exercise (Fat burn 30 min) of weak strength.

Furthermore, information about a running distance and time that may be challengeable for each step and information about safe exercise conditions (e.g., run speed) according to a disease type may be displayed in the “Run method” item.

In an “Optimizing Program” item, a group to which the examinee belongs based on the calculated cardiopulmonary capacity index of the examinee is indicated. Information about an exercise program suitable for the group of the examinee is provided.

More specifically, the group of the examinee may be determined based on the calculated ratio of oxygen utilization (Mets). For example, when the ratio of oxygen utilization (Mets) is less than “10”, the group may be classified as “Beginner.” When the ratio of oxygen utilization (Mets) is “10” to “12”, the group may be classified as “Intermediate.” When the ratio of oxygen utilization (Mets) is more than “12”, the group may be classified as “Advanced.”

In the “Optimizing Program” item, information about exercise programs corresponding to “Beginner”, “Intermediate” and “Advanced”, respectively, may be indicated.

In the case of “Beginner”, an exercise program in which running is performed at a run speed corresponding to 60% of the calculated pace for 30 minutes is indicated. An increase of the run speed by 0.3 km/h upon success may be suggested.

In the case of “Intermediate”, an exercise program in which running is performed at a run speed corresponding to 80% of the pace is indicated. A different run time may be suggested depending on a caloric value to be consumed.

In the case of “Advanced”, an exercise program in which running is repeated at a run speed corresponding to 100% of the pace for 2 minutes and then a run speed corresponding to 60% of the pace for 2 minutes is indicated. A different repetition number may be suggested depending on a caloric value to be consumed.

In accordance with the method of providing an exercise test service according to an embodiment of the present invention, test result values measured after running at a specific distance or time in an indoor exercise apparatus, such as a treadmill are input, and a diagnosis reference value indicative of the cardiopulmonary capacity of an examinee is provided. Accordingly, objectified data for cardiopulmonary capacity of an examinee can be provided by inputting only test result values without using an expensive test apparatus.

Furthermore, an examinee is classified as one of a plurality of groups based on a diagnosis reference value calculated using input result values, and information about an exercise program corresponding to the classified group is provided. Accordingly, a program including exercise conditions, such as a run speed and distance optimized for a user, can be suggested.

The method of providing an exercise test service according to an embodiment of the present invention may be implemented by an exercise test system to be described below. A description of configurations and operations that belong to the configurations and operations of the exercise test system and that are the same as those described with reference to FIGS. 1 to 6 is omitted.

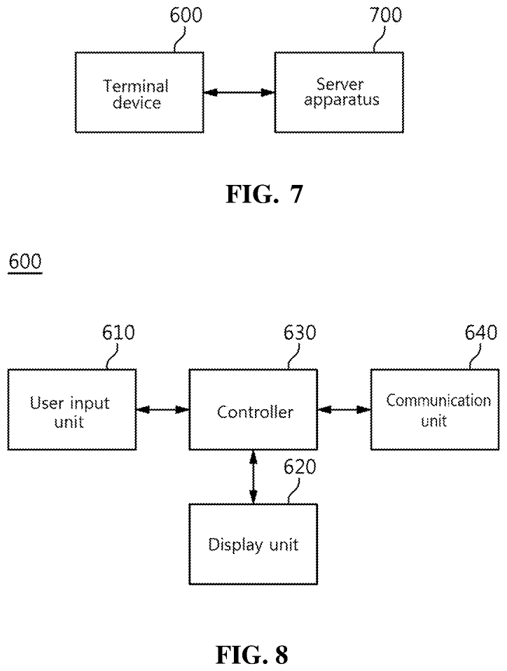

FIG. 7 is a schematic block diagram of the configuration of the exercise test service system according to an embodiment of the present invention. The exercise test service system may include a terminal device 600 and a server apparatus 700.

Referring to FIG. 7, the terminal device 600 may perform the method of providing an exercise test service, described with reference to FIGS. 1 to 6, solely or may perform the method while operating in conjunction with the server apparatus 700.

For example, the terminal device 600 may receive test result values measured when an examinee performs a test of a specific condition, may calculate at least one diagnosis reference value indicative of the cardiopulmonary capacity of the examinee using the input result values, may classify the examinee as one of a plurality of groups based on the calculated diagnosis reference value, and may provide information about an exercise program corresponding to the classified group.

The terminal device 600 functions to receive test result values measured when an examinee performs a test of a specific condition and to display a diagnosis reference value calculated based on the test result values and information about an exercise program corresponding to a group of the examinee. A function of calculating at least one diagnosis reference value indicative of the cardiopulmonary capacity of the examinee using the input result values and classifying the examinee as one of a plurality of groups may be performed by the server apparatus 700.

The terminal device according to an embodiment of the present invention may be included in a desktop computer, a mobile phone, a smart phone, a laptop computer, a terminal for digital broadcasting, personal digital assistants (PDA), a portable multimedia player (PMP), or a navigator, but the present invention is not limited thereto. In addition, the terminal device may include various devices capable of user input and information indication.

Referring to FIG. 8, the terminal device 600 may include a user input unit 610, a display unit 620, a controller 630 and a communication unit 640.

The user input unit 610 of the terminal device 600 receives test result values measured when an examinee performs a test of a specific condition.

The display unit 620 may display at least one diagnosis reference value indicative of the cardiopulmonary capacity of the examinee calculated using the input result values, and may display information about an exercise program corresponding to a group that belongs to a plurality of groups and to which the examinee belongs based on the diagnosis reference value.

The controller 630 may perform control so that result values received through the user input unit 610 are transmitted to the server apparatus 700 through the communication unit 640 and information about an exercise program corresponding to a group to which an examinee belongs, which is received from the server apparatus 700 through the communication unit 640, is displayed on the display unit 620. The controller 630 may function to control an overall operation of the terminal device 600.

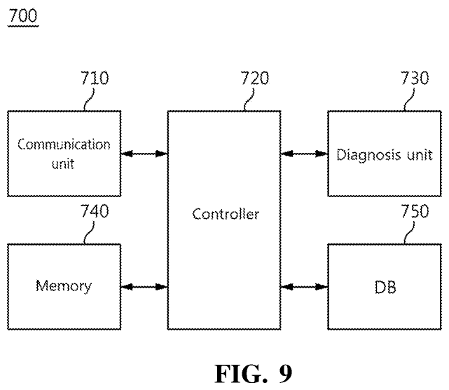

Referring to FIG. 9, the server apparatus 700 may include a communication unit 710, a controller 720, a diagnosis unit 730, memory 740 and a database (DB) 750.

The server apparatus 700 includes the communication unit 710 connected to the terminal device 600 over a communication network in order to provide an exercise test service. The communication unit 710 may receive test result values measured when an examinee performs a test of a specific condition from the terminal device 600.

The controller 720 transfers the test result values, received through the communication unit 710, to the diagnosis unit 730. The diagnosis unit 730 calculates at least one diagnosis reference value indicative of the cardiopulmonary capacity of the examinee using the received result values.

Furthermore, the diagnosis unit 730 may classify the examinee as one of a plurality of groups based on the calculated diagnosis reference value.

The memory 740 stores information about a plurality of exercise programs respectively corresponding to a plurality of groups to which the examinee will belong. The plurality of exercise programs may have different exercise conditions (e.g., a run speed and time).

The controller 720 may configure an exercise program corresponding to a group to which an examinee belongs using information stored in the memory 740, and may perform control so that information about exercise conditions included in the configured exercise program is transmitted to the terminal device 600 through the communication unit 710.

In accordance with another embodiment of the present invention, an examinee can manage his or her health based on objective data because a change in the physical fitness level of the examinee is measured based on a cardiopulmonary capacity value obtained by performing an exercise test and the rate of a disease of the examinee is predicted and provided.

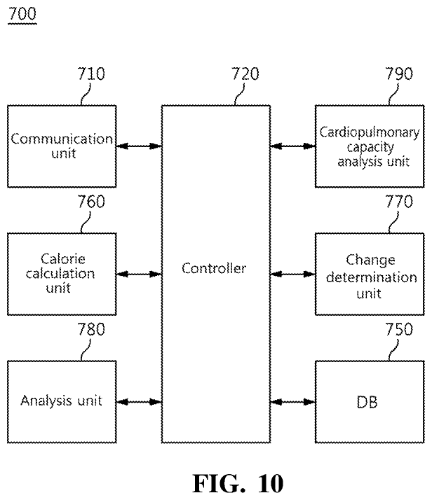

FIG. 10 is a block diagram showing another embodiment of the configuration of the server apparatus shown in FIG. 7. The server apparatus 700 may include a communication unit 710, a controller 720, a cardiopulmonary capacity analysis unit 790, a DB 750, a calorie calculation unit 760, a change determination unit 770 and an analysis unit 780. A description of elements that belong to the elements of the server apparatus 700 of FIG. 10 and that are the same as those described with reference to FIGS. 8 and 9 is omitted hereunder.

Referring to FIG. 10, the cardiopulmonary capacity analysis unit 790 provides information about an exercise program suitable for an examinee based on a cardiopulmonary capacity value of the examinee obtained by performing an exercise test.

A method of measuring maximum oxygen consumption (VO2max) or the ratio of oxygen utilization (Mets), that is, a cardiopulmonary capacity value of the examinee, through the exercise test and a method of configuring an exercise program suitable for the examinee based on the measured cardiopulmonary capacity value may be the same as those described with reference to FIGS. 1 to 9.

The calorie calculation unit 760 calculates weekly calorie consumption of an examinee based on calorie consumption and the number of exercises per week corresponding to an exercise program suggested by the cardiopulmonary capacity analysis unit 790.

For example, maximum oxygen consumption (VO2max) measured through an exercise test may indicate the physical fitness level of an examinee. The training volume of the examinee, that is, a proper caloric value that may be consumed through one exercise, may be determined based on the maximum oxygen consumption (VO2max).

A corresponding exercise program may be suggested to the examinee through an exercise test based on the training volume (i.e., proper calorie consumption) of the examinee.

To this end, the exercise program suggested to the examinee includes a plurality of exercise conditions corresponding to a plurality of pieces of calorie consumption, respectively. The exercise conditions may include a run speed and distance that needs to be performed by the examinee through once exercise.

Weekly calorie consumption of the examinee that may be consumed a week through the exercise program may be calculated by multiplying the calculated calorie consumption of the examinee by the number of exercises per week.

For example, if an examinee has a physical fitness level at which 180 kcal can be consumed through one exercise based on maximum oxygen consumption (VO2max) measured through an exercise test and the examinee can take two exercises a week, weekly calorie consumption of the examinee is 360 kcal/week.

Maximum oxygen consumption (VO2max) and weekly calorie consumption of an examinee before an exercise program starts may be stored and managed in the DB 750.

The change determination unit 770 determines a change in the weekly calorie consumption of the examinee using a cardiopulmonary capacity value of the examinee obtained by performing the exercise test again after the exercise program is performed for a specific period.

For example, when maximum oxygen consumption (VO2max) of an examinee is measured through an exercise test and an exercise program suitable for the examinee is suggested based on the maximum oxygen consumption (VO2max), the examinee takes exercises based on the suggested exercise program.

After the examinee performs the suggested exercise program for a specific period, maximum oxygen consumption (VO2max) of the examinee may be measured again through an exercise test, and weekly calorie consumption of the examinee may be calculated again.

In this case, the change determination unit 770 may calculate a change in the weekly calorie consumption by comparing past weekly calorie consumption stored in the DB 750 in relation to the examinee with the weekly calorie consumption of the examinee calculated after performing the exercise program for a specific period.

Thereafter, the analysis unit 780 determines whether at least one of a plurality of guidelines related to the weekly calorie consumption is satisfied or an arrival ratio based on a change in the weekly calorie consumption of the examinee determined by the change determination unit 770.

For example, the plurality of guidelines may include reference values, such as weekly calorie consumption for chronic disease prevention, weekly calorie consumption for obesity prevention, weekly calorie consumption for obesity improvement, and weekly calorie consumption for lipid plaque removal. Information about corresponding reference values is stored in the DB 750.

Information about the current state of an examinee regarding a guideline related to weekly calorie consumption determined by the analysis unit 780 as described above may be transmitted to the terminal device 600 through the communication unit 710 and transmitted to the examinee.

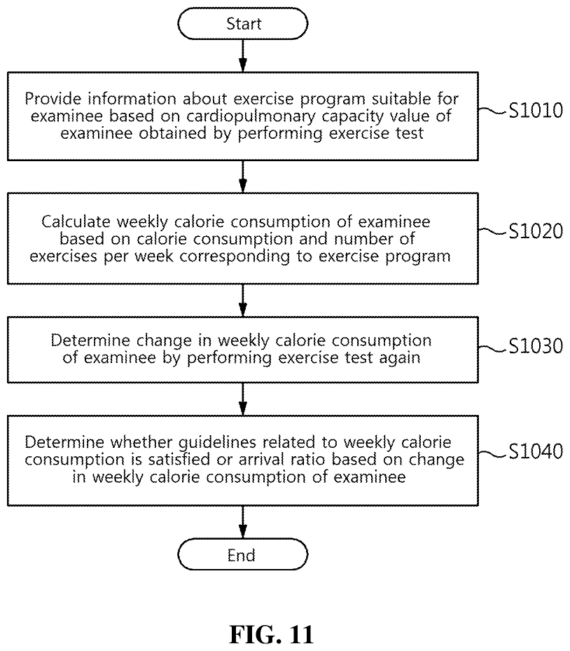

Embodiments of a method of analyzing a change in the physical fitness level of an examinee while operating in conjunction with an exercise test and providing a guideline for health management are described in detail below with reference to FIG. 11.

FIG. 11 is a flowchart illustrating a method of providing a health management service while operating in conjunction with an exercise test according to an embodiment of the present invention. The service provision method is described in connection with the block diagram showing the configuration of the server apparatus 700 of FIG. 10.

Referring to FIG. 11, the cardiopulmonary capacity analysis unit 790 of the server apparatus 700 configures an exercise program suitable for an examinee based on a cardiopulmonary capacity value of the examinee obtained by performing an exercise test, and provides information about the exercise program (step S1010).

As described above with reference to FIGS. 1 to 9, the cardiopulmonary capacity value may be maximum oxygen consumption (VO2max) or the ratio of oxygen utilization (Mets) calculated using test result values measured when the examinee runs a specific distance or for a specific time.

Furthermore, the exercise program suggested to the examinee includes one or more exercise conditions according to calorie consumption. The exercise conditions may include at least one of a run speed and distance suggested to the examinee.

The calorie calculation unit 760 calculates weekly calorie consumption of the examinee based on calorie consumption and the number of exercises per week corresponding to the suggested exercise program (step S1020).

For example, one of a plurality of exercise programs having different calorie consumption based on a physical fitness level of the examinee according to maximum oxygen consumption (VO2max) measured through an exercise test may be suggested to the examinee. Weekly calorie consumption of the examinee may be calculated by multiplying calorie consumption consumed by performing the suggested exercise program once and the number of times that the examinee can take exercise a week.

If the examinee performs the suggested exercise program for a specific period, maximum oxygen consumption (VO2max) of the examinee may rise and thus a physical fitness level of the examinee rises. As a result, a caloric value that may be consumed through one exercise may be increased.

Furthermore, when the examinee continues to perform the exercise program, the number of times that the examinee can take exercise a week may also increase. Accordingly, weekly calorie consumption of the examinee may further increase.

After steps S1010 and S1020, the examinee may perform the suggested exercise program for a specific period (e.g., 1 month, 2 months or 12 weeks) based on his or her cardiopulmonary capacity value, may perform an exercise test again after a lapse of the specific period, and may check his or her cardiopulmonary capacity value.

The change determination unit 770 determines a change in the weekly calorie consumption of the examinee using a cardiopulmonary capacity value of the examinee obtained by performing the exercise test again after the examinee performs the exercise program for a specific period (step S1030).

To this end, maximum oxygen consumption (VO2max) of the examinee, calorie consumption and weekly calorie consumption when one exercise is performed may be cumulatively stored in the DB 750 when the examinee performs an exercise test.

The analysis unit 780 determines whether at least one of a plurality of guidelines related to the weekly calorie consumption is satisfied or an arrival ratio based on a change in the weekly calorie consumption of the examinee determined at step S1030 (step S1040).

The guidelines related to the weekly calorie consumption may include reference values, such as weekly calorie consumption for chronic disease prevention, weekly calorie consumption for obesity prevention, weekly calorie consumption for obesity improvement, and weekly calorie consumption for lipid plaque removal.

For example, a minimum guideline for preventing a chronic disease may be based on weekly calorie consumption of 1000 kcal/week. Guidelines, such as 1500 kcal/week in order to prevent obesity, 2000 kcal/week in order to improve obesity, and 2200 kcal/week in order to remove a lipid plaque, that is, the cause of a cardiovascular patient, may have been stored in the DB 750.

However, the present invention is not limited to the guidelines. Guidelines for suggesting a criterion for weekly calorie consumption in relation to various diseases, such as diabetes, cholesterol, blood pressure, and a metabolic syndrome, may have been stored in the DB 750.

The server apparatus 700 transmits information about a change in the arrival ratio for a guideline related to the weekly calorie consumption according to a change in the weekly calorie consumption of the examinee described through the communication unit 710 to the terminal device 600 so that the information is displayed on a screen of the terminal device 600.

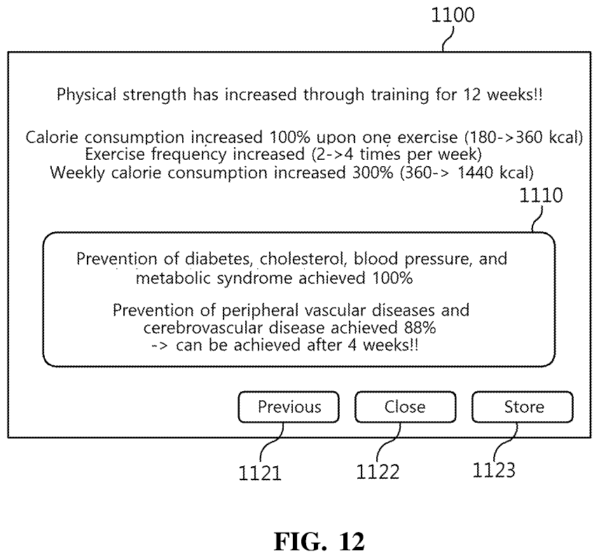

FIG. 12 is a diagram for illustrating embodiments of a method of providing a user with a change in the physical fitness level of an examinee through an exercise program and information about corresponding effects. FIG. 12 shows an example of the configuration of a screen displayed in the terminal device 600 of an examinee (or a person to be tested).

Referring to FIG. 12, the examinee receives an optimal exercise program suggested through an exercise test, takes exercise for 12 weeks, and performs an exercise test again. The results of the execution of the exercise test are analyzed, and the analyzed information may be displayed on the screen 1100 of the terminal device 600 while operating in conjunction with the server apparatus 700.

If the examinee has a physical fitness level at which 180 kcal can be consumed through one exercise based on maximum oxygen consumption (VO2max) measured through an exercise test and the examinee can take two exercises a week, weekly calorie consumption of the examinee is 360 kcal/week.

After the examinee performed an exercise program suggested based on the physical fitness level for 12 weeks, maximum oxygen consumption (VO2max) of the examinee was measured through an exercise test. As a result of the measurement, it can be seen that a caloric value that may be consumed by the examinee through one exercise has been increased to 360 kcal 100%, the number of exercises per week has been increased to four times, and thus weekly calorie consumption has been increased to 1440 kcal/week 300%.

In this case, information about a change in the calorie consumption when the examinee takes exercise once, a change in the number of exercises per week, and a change in the weekly calorie consumption may be received from the server apparatus 700 and may be displayed on the screen 1100 of the terminal device 600.

Furthermore, information indicating that 1000 kcal/week, that is, a guideline for preventing chronic diseases such as diabetes, cholesterol, blood pressure, and a metabolic syndrome, has been achieved and a guideline for preventing peripheral vascular diseases and a cerebrovascular disease has been achieved about 88% due to an increase of the physical fitness level of the examinee and an increase of the weekly calorie consumption through the exercise program may be displayed in the lower region 1110 of the screen 1100.

In accordance with another embodiment of the present invention, the server apparatus 700 may estimate a period necessary to satisfy a specific guideline using the exercise program of an examinee and a change in the weekly calorie consumption, and may provide a user with information about the estimated period by transmitting the information to the terminal device 600 through the communication unit 710.

For example, estimation information indicating that a guideline for preventing peripheral vascular diseases and a cerebrovascular disease can be achieved if an examinee performs an exercise program for four weeks may be displayed in the lower region 1110 of the screen 1100.

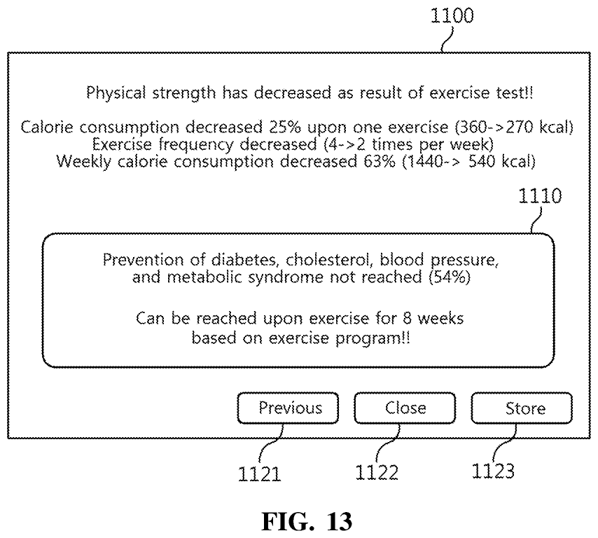

Furthermore, as shown in FIG. 13, as a result of the re-execution of an exercise test after a lapse of a specific period, maximum oxygen consumption (VO2max) of an examinee may be decreased. Accordingly, it may be seen that a caloric value that may be consumed by the examinee through one exercise has decreased to 270 kcal 25%, the number of exercises per week has been reduced to two times, and thus weekly calorie consumption has decreased to 540 kcal/week 63%.

In this case, information about the decrement of calorie consumption when the examinee takes exercise once due to the shortage of exercise and the decrement of weekly calorie consumption may be received from the server apparatus 700 and displayed on the screen 1100 of the terminal device 600.

Furthermore, information indicating that a guideline for preventing chronic diseases, such as diabetes, cholesterol, blood pressure, and a metabolic syndrome, has not reached 1000 kcal/week due to a reduction of the physical fitness level of the examinee through the exercise program and a reduction of corresponding weekly calorie consumption may be displayed in the lower region 1110 of the screen 1100.

Furthermore, if an examinee faithfully performs an exercise program suggested through an exercise test, the server apparatus 700 may estimate a period necessary to satisfy a chronic disease prevention guideline, and may provide a user with information about the period by transmitting the information to the terminal device 600 through the communication unit 710.

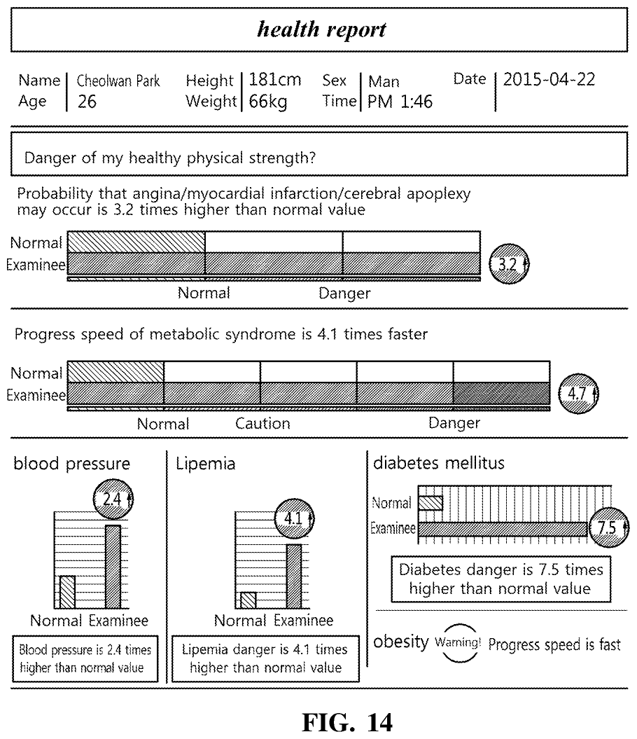

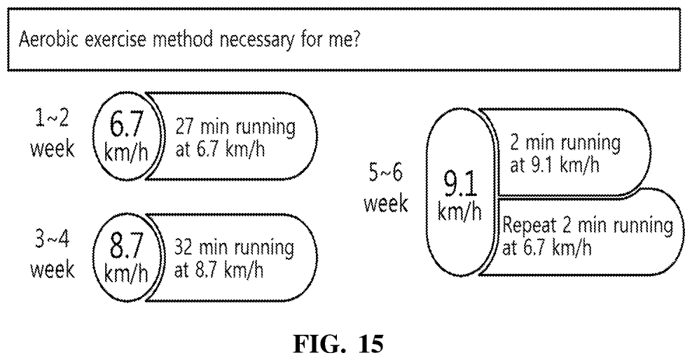

Various types of information, such as a physical fitness level of an examinee analyzed as described above while operating in conjunction with an exercise test, the probability that a disease may occur and the process speed and normal value versus ratio of the diseases, and an exercise method suggested to the examinee may be provided to a user in the form of the health report shown in FIGS. 14 and 15. The health report has been separately shown in FIGS. 14 and 15, but has one sheet of a report in which the contents of FIGS. 14 and 15 have been combined.

Furthermore, an examinee can manage health based on objective data because the rate of a disease of the examinee is estimated based on a cardiopulmonary capacity value and a change in the results of medical check-up of the examinee and provided.

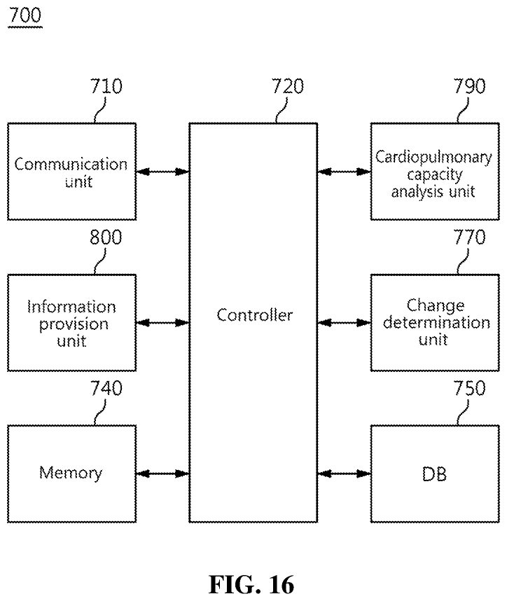

FIG. 16 is a block diagram showing another embodiment of the configuration of the server apparatus shown in FIG. 7. The server apparatus 700 may include a communication unit 710, a controller 720, a cardiopulmonary capacity analysis unit 790, memory 740, a DB 750, an information provision unit 800 and a change determination unit 770. A description of elements that belong to the elements of the server apparatus 700 of FIG. 16 and that are the same as those described with reference to FIGS. 8 and 9 is omitted hereunder.

Referring to FIG. 16, the cardiopulmonary capacity analysis unit 790 provides information about an exercise program suitable for an examinee based on a cardiopulmonary capacity value of the examinee obtained by performing an exercise test.

A method of measuring maximum oxygen consumption (VO2max) or the ratio of oxygen utilization (Mets), that is, a cardiopulmonary capacity value of the examinee through the exercise test and a method of configuring an exercise program suitable for the examinee based on the measured cardiopulmonary capacity value have be the same as those described with reference to FIGS. 1 to 9.

The change determination unit 770 determines a change in the cardiopulmonary capacity value by comparing a cardiopulmonary capacity value of an examinee with a cardiopulmonary capacity value of the examinee obtained by performing the exercise test again after the exercise program is performed for a specific period, and determines a change in the results of medical check-up by comparing the health records of the examinee with the health records of the examinee obtained by performing medical check-up again after a lapse of a specific period.

For example, when maximum oxygen consumption (VO2max) of the examinee is measured through an exercise test and an exercise program suitable for the examinee is suggested based on the maximum oxygen consumption, the examinee takes exercise based on the suggested exercise program.

The health records (e.g., the results of a blood test and the results of a urine test) of the examinee may be stored in the memory 740 or the DB 750 before the examinee starts the exercise program.

The health records of the examinee may be received from the terminal device 600 or another server (not shown) through the communication unit 710.

After the examinee performs the suggested exercise program for a specific period, maximum oxygen consumption (VO2max) of the examinee is measured again through an exercise test. The health records (e.g., the results of a blood test and the results of a urine test) of the examinee are received again through the communication unit 710.

The received health records of the examinee may be the results of medical check-up received after the examinee has taken exercise based on the suggested exercise program for a specific period.

Furthermore, a change in the results of medical check-up and a change in the rate of one or more diseases have been stored in the memory 740 in a corresponding manner.

For example, data indicative of association between the results of a blood test and the rate of a specific disease and data indicative of association between the results of a urine test and the rate of a specific disease may have been previously stored in the memory 740 or the DB 750.

The information provision unit 800 provides information about a change in the rate of a disease corresponding to a change in the results of medical check-up of an examinee in connection with at least one of a change in a cardiopulmonary capacity value and a change in the result of medical check-up.

For example, information about the increment of maximum oxygen consumption (VO2max) of an examinee, a change in the items as the results of a blood test or a urine test of the examinee, the decrement of the rate of a specific disease of the examinee, and a change in the mortality rate of the examinee, that is, effects obtained by performing an exercise program for a specific period, may be transmitted to the terminal device 600 through the communication unit 710 and provided to a user.

Furthermore, the server apparatus 700 may further include an estimation unit (not shown) for estimating a period necessary for the results of medical check-up to arrive a normal range and a period necessary for the rate of a specific disease to decrease to a preset reference value based on a change in the cardiopulmonary capacity value or a change in the results of medical check-up.

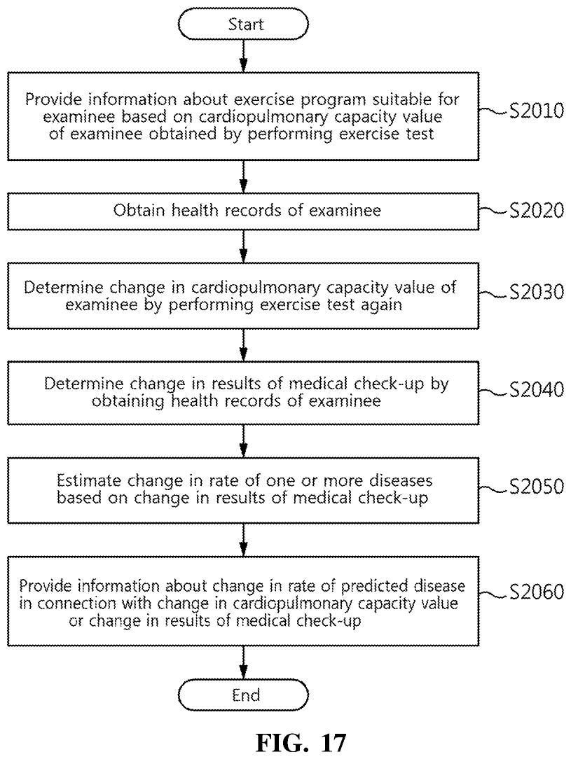

Embodiments of a method of analyzing health records while operating in conjunction with an exercise test are described in detail below with reference to FIGS. 17 to 19.

FIG. 17 shows a method of providing a health record analysis service while operating in conjunction with an exercise test according to an embodiment of the present invention in a flowchart form. The service provision method is described in connection with the block diagram showing the configuration of the server apparatus 700 of FIG. 16.

Referring to FIG. 17, the cardiopulmonary capacity analysis unit 790 of the server apparatus 700 configures an exercise program suitable for an examinee based on a cardiopulmonary capacity value of the examinee obtained by performing an exercise test, and provides information about the exercise program (step S2010).

As described above with reference to FIGS. 1 to 9, the cardiopulmonary capacity value may be maximum oxygen consumption (VO2max) or the ratio of oxygen utilization (Mets) calculated using test result values measured when the examinee runs a specific distance or for a specific time.

Furthermore, the exercise program suggested to the examinee includes one or more exercise conditions according to calorie consumption. The exercise conditions may include at least one of a run speed and distance suggested to the examinee.

The controller 720 obtains the health records of the examinee by receiving it from the terminal device 600 or another server (not shown) through the communication unit 710 (step S2020).

For example, the health records include the results of a blood test value including at least one of a fasting blood glucose level, blood pressure, a cholesterol value and a neutral fat value and the results of a urine test value including glycated hemoglobin (HbA1C). However, the present invention is not limited to the examples and may include the results of check-up of various items in addition to the results of a blood test and the results of a urine test.

After steps S2010 and S2020, the examinee may check his or her cardiopulmonary capacity value by performing the suggested exercise program for a specific period (e.g., 1 month, 2 months or 12 weeks) based on the cardiopulmonary capacity value and performing an exercise test again for a specific period, and may check his or her health through medical check-up.

The change determination unit 770 determines a change in the cardiopulmonary capacity value by comparing the cardiopulmonary capacity value of the examinee with the cardiopulmonary capacity value of the examinee obtained by performing the exercise test again for a specific period after the exercise program is performed (step S2030).

Furthermore, the change determination unit 770 determines a change in the results of medical check-up by comparing the health records of the examinee with the health records of the examinee obtained by performing medical check-up again after a lapse of a specific period (step S2040).

To this end, corresponding results (e.g., maximum oxygen consumption (VO2max) and health records of the examinee) may have been cumulatively stored in the memory 740 or the DB 750 in connection with personal information of the examinee whenever an exercise test or medical check-up is performed.

The controller 720 estimates a change in the rate of one or more specific diseases based on a change in the results of medical check-up (step S2050), and provides information about a change in the rate of a predicted disease in connection with a change in the cardiopulmonary capacity value or a change in the results of medical check-up based on the result of the execution of the exercise program for a specific period (step S2060).

Association between a change in the results of medical check-up and a change in the rate of a specific disease for estimating a change in the rate of a specific disease from a change in the results of medical check-up may have been stored in the memory 740 or the DB 750.

For example, in the case of the results of a blood test, such as a fasting blood glucose level, blood pressure, cholesterol value and neutral fat value, a look-up table in which a change in the results of a blood test value corresponds to a change in the rate of obesity, diabetes, hyperlipidemia and heart-cerebrovascular diseases may have been stored in the memory 740.

Alternatively, in the case of the results of a urine test such as glycated hemoglobin (HbA1C), a look-up table in which a change in the results of a urine test value corresponds to a change in the rate of myocardial infarction, cerebral apoplexy, diabetes, peripheral vascular diseases and micro vascular diseases may have been stored in the memory 740.

In accordance with another embodiment of the present invention, the server apparatus 700 may estimate a period necessary for the results of medical check-up to reach a normal range based on a change in the cardiopulmonary capacity value or a change in the results of medical check-up or a period necessary for the rate of a disease to decrease to a reference value or less, and may provide a user with information about the period by transmitting the information to the terminal device 600 through the communication unit 710.

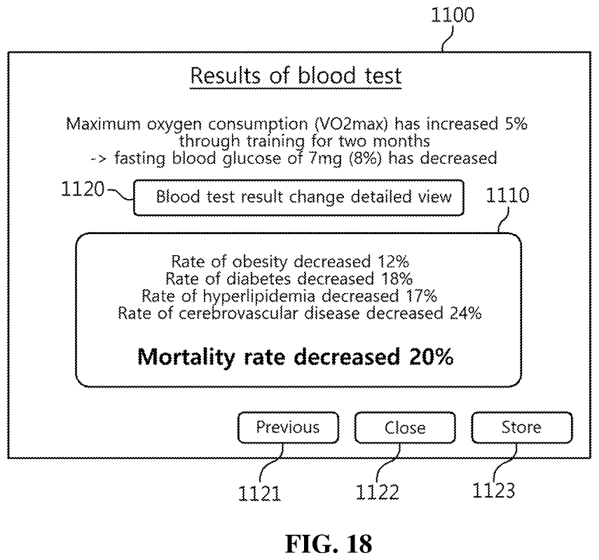

FIG. 18 is a diagram for illustrating an embodiment of a method of providing an examinee with the analysis of the results of a blood test. FIG. 18 shows an example of the configuration of a screen displayed in the terminal device 600 of an examinee (or a person to be tested).

Referring to FIG. 18, the examinee performs an exercise test and medical check-up after taking exercise for two months based on an optimal exercise program suggested through an exercise test. Information obtained by analyzing the results of the exercise test and medical check-up may be displayed on the screen 1100 of the terminal device 600 while operating in conjunction with the server apparatus 700.

It can be seen that as the results of exercise of the examinee for two months, maximum oxygen consumption (VO2max) of the examinee has increased 5% through the exercise test.

Furthermore, it can be seen that fasting blood glucose of 7 mg has decreased and blood glucose of about 8% has decreased compared to the results of medical check-up prior to the results of medical check-up due to an increase of the maximum oxygen consumption (VO2max) of the examinee.

When “Blood test results change detailed view” button 1120, information indicating that in blood pressure, synizesis and diastole have decreased 3 mmhg (4%), total cholesterol has decreased 6 mg/dl (6%), HDL cholesterol has decreased 4 mg/dl (5%), LDL cholesterol has decreased 7 mg/dl (8%), and neutral fat has decreased 12 mg/dl (10%) in addition to blood glucose may be displayed on the screen 1100.

Furthermore, disease-related information, indicating that the rate of obesity has decreased 12%, the rate of diabetes has decreased 18%, the rate of hyperlipidemia has decreased 17%, and the rate of a cerebrovascular disease has decreased 24% due to an increase of the maximum oxygen consumption (VO2max) and a change in the medical check-up of the examinee and a mortality rate attributable to each of the diseases has decreased 20%, may be displayed in the lower region 1110 of the screen 1100.

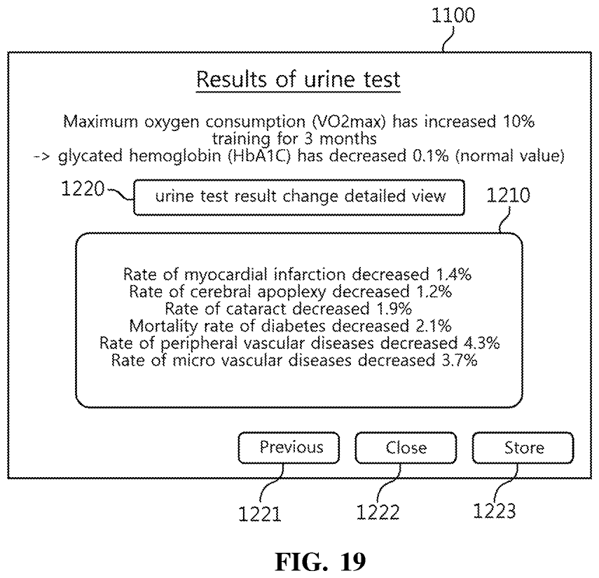

FIG. 19 is a diagram for illustrating an embodiment of a method of providing an examinee with the analysis of the results of a urine test. FIG. 19 shows an example of the configuration of a screen displayed on the terminal device 600 of an examinee (or a person to be tested).

Referring to FIG. 19, the examinee performs an exercise test and medical check-up after taking exercise for three months based on an optimal exercise program suggested through an exercise test. Information obtained by analyzing the results of the exercise test and medical check-up may be displayed on the screen 1100 of the terminal device 600 while operating in conjunction with the server apparatus 700.

It can be seen that as the results of exercise of the examinee for three months, maximum oxygen consumption (VO2max) of the examinee has increased 10% through the exercise test.

Furthermore, it can be seen that due to an increase of the maximum oxygen consumption (VO2max) of the examinee, glycated hemoglobin (HbA1C) has decreased 0.1% and reached a normal value compared to the results of medical check-up prior to the results of medical check-up.

Furthermore, disease-related information, indicating that the rate of myocardial infarction has decreased 1.4%, the rate of cerebral apoplexy has decreased 1.2%, the rate of cataract has decreased 1.9%, a mortality rate of diabetes has decreased 2.1%, the rate of peripheral vascular diseases has decreased 4.3%, and the rate of micro vascular diseases has decreased 3.7% due to an increase of maximum oxygen consumption (VO2max) and increase of glycated hemoglobin (HbA1C) of the examinee, may be displayed in the lower region 1210 of the screen 1100.

In accordance with an embodiment of the present invention, test result values measured when an examinee runs a specific distance or for a specific time in an indoor exercise apparatus such as a treadmill, are input, and a diagnosis reference value indicative of the cardiopulmonary capacity of the examinee is provided. Accordingly, objectified data for the cardiopulmonary capacity of the examinee can be provided by inputting only the test result values without using an expensive test apparatus.

Furthermore, a cost for an exercise test can be reduced and examinees' convenience can be improved because an exercise test performed outdoors can be performed in an indoor exercise apparatus conveniently.

Furthermore, a program including exercise conditions, such as a run speed and distance optimized for a user, can be provided to the user because an examinee is classified as one of a plurality of groups based on a diagnosis reference value obtained using input result values and information about an exercise program corresponding to the classified group is provided.

In accordance with another embodiment of the present invention, an examinee can manage health based on objective data because a change in the physical fitness level of the examinee is measured based on a cardiopulmonary capacity value obtained by performing an exercise test and the rate of a disease of the examinee is estimated and provided.

In accordance with another embodiment of the present invention, the exercise of an examinee can be induced effectively because a change in a cardiopulmonary capacity value of the examinee obtained by performing an exercise test and a change in the results of medical check-up are analyzed in conjunction with each other.

Furthermore, an examinee can manage health based on objective data because the rate of a disease of the examinee is estimated and provided based on a cardiopulmonary capacity value and a change in the results of medical check-up of the examinee.

The aforementioned method according to the present invention may be produced in the form of a program to be executed by a computer and may be stored in a computer-readable recording medium. The computer-readable recording medium may include ROM, RAM, CD-ROM, a magnetic tape, a floppy disk, and an optical data storage device, for example. Furthermore, the computer-readable recording medium includes media implemented in the form of carrier waves (e.g., transmission through the Internet).

Furthermore, the computer-readable recording medium may be distributed to computer systems connected over a network, and computer-readable code may be stored and executed in a distributed manner. Furthermore, a functional program, code and code segments for implementing the method may be easily reasoned by programmers those skilled in the art to which the present invention pertains.

Furthermore, although the preferred embodiments of the present invention have been illustrated and described above, the present invention is not limited to the aforementioned specific embodiments, and may be modified in various ways by a person having ordinary skill in the art to which the present invention pertains without departing from the gist of the present invention claimed in the claims. Such modified embodiments should not be individually understood from the technical spirit or prospect of this specification.

1. A method of providing a health management service while operating in conjunction with an exercise test, the method comprising:
providing information about an exercise program suitable for an examinee based on a cardiopulmonary capacity value of the examinee obtained by performing an exercise test;
calculating weekly calorie consumption of the examinee based on calorie consumption and a number of exercises per week corresponding to the exercise program;
determining a change in the weekly calorie consumption of the examinee based on a cardiopulmonary capacity value of the examinee obtained by performing the exercise test again after the exercise program for a specific period; and
determining whether at least one of a plurality of guidelines related to the weekly calorie consumption is satisfied or an arrival ratio based on the determined change in the weekly calorie consumption of the examinee.

2. The method of claim 1, wherein the cardiopulmonary capacity value comprises maximum oxygen consumption (VO2max) or a ratio of oxygen utilization (Mets) calculated based on test result value measured when the examinee runs a specific distance or for a specific time.

3. The method of claim 1, wherein the exercise program comprises a plurality of exercise conditions respectively corresponding to a plurality of calorie consumptions.

4. The method of claim 3, wherein the exercise conditions comprises at least one of a run speed and distance suggested to the examinee.

5. The method of claim 1, wherein the plurality of guidelines comprises at least one of weekly calorie consumption for chronic disease prevention, weekly calorie consumption for obesity prevention, weekly calorie consumption for obesity improvement, and weekly calorie consumption for lipid plaque removal.

6. The method of claim 1, further comprising displaying a change of an arrival ratio for the guidelines related to the weekly calorie consumption according to a change in the weekly calorie consumption of the examinee on a screen of a terminal device.

7. The method of claim 1, further comprising estimating a period necessary to satisfy at least one of the plurality of guidelines based on a change in the weekly calorie consumption of the examinee.

8. A recording medium on which a program for executing a method according to claim 1 in a computer has been recorded.

9. A server for providing a health management service while operating in conjunction with an exercise test, the server comprising:
a cardiopulmonary capacity analysis unit configured to provide information about an exercise program suitable for an examinee based on a cardiopulmonary capacity value of the examinee obtained by performing an exercise test;
a calorie calculation unit configured to calculate weekly calorie consumption of the examinee based on calorie consumption and a number of exercises per week corresponding to the exercise program;
a change determination unit configured to determine a change in the weekly calorie consumption of the examinee based on a cardiopulmonary capacity value of the examinee obtained by performing the exercise test again after the exercise program for a specific period; and
an analysis unit configured to determine whether at least one of a plurality of guidelines related to the weekly calorie consumption is satisfied or an arrival ratio based on the determined change in the weekly calorie consumption of the examinee.

10. The server of claim 9, wherein the cardiopulmonary capacity value comprises maximum oxygen consumption (VO2max) or a ratio of oxygen utilization (Mets) calculated based on test result value measured when the examinee runs a specific distance or for a specific time.

11. The server of claim 9, wherein the exercise program comprises a plurality of exercise conditions respectively corresponding to a plurality of calorie consumptions.

12. The server of claim 11, wherein the exercise conditions comprises at least one of a run speed and distance suggested to the examinee.

13. The server of claim 11, wherein the plurality of guidelines comprises at least one of weekly calorie consumption for chronic disease prevention, weekly calorie consumption for obesity prevention, weekly calorie consumption for obesity improvement, and weekly calorie consumption for lipid plaque removal.
